(19) United States
(12) Patent Application Publication
(10) Pub. No.: US 2016/0183878 A1
(43) Pub. Date: Jun. 30, 2016

(54) TECHNOLOGIES FOR MANAGING DEVICE FUNCTIONS OF AN INGESTIBLE COMPUTING DEVICE

(71) Applicants: John C. Weast, Portland, OR (US); Brian C. Johnson, Portland, OR (US); Tobias M. Kohlenberg, Portland, OR (US)

(72) Inventors: John C. Weast, Portland, OR (US); Brian C. Johnson, Portland, OR (US); Tobias M. Kohlenberg, Portland, OR (US)

(21) Appl. No.: 14/583,705

(22) Filed: Dec. 27, 2014

(51) Int. Cl. A61B 5/00; A61B 5/07

(52) U.S. Cl. CPC A61B 5/6861; A61B 5/073; A61B 5/4839; A61B 5/4848; A61B 5/746; A61B 5/7221; A61B 2560/0475; A61B 2562/08

(57) ABSTRACT

Technologies for managing device functions of an ingestible computing device include determining whether adaptation of a device function of the ingestible computing device is required based on sensor data generated by the ingestible computing device. If device function adaption is determined to be required, the ingestible computing device determines a new device function to be performed, obtains program code modules to perform the new device function, and locally compiles the new program code modules to generate executable code. The ingestible computing device may execute the executable code to cause the ingestible computing device to begin performing the new device function. The ingestible computing device may obtain the program code modules from a local function database or via an authenticated wireless communication.

### BACKGROUND

Ingestible computing devices are generally small form factor computational devices capable of being swallowed, implanted, or otherwise ingested into a user. Ingestible computing devices may be embodied as various types of devices, including digital pills, smart implants, artificial organs, other biological components. Ingestible computing devices oftentimes include one or more sensors for detecting aspects of the internal environment of the user.

Ingestible computing devices are commonly fabricated to perform a particular function, such as sensing a biological function of the user or detecting efficacy of a drug. However, depending on the ingestible computing device, the programmed function, and/or conditions of the user, the programmed function of the ingestible computing device may become obsolete or undesirable. In such situations, the ingestible computing device is removed from the user. For example, some ingestible computing devices are designed to operate for only a defined period of time (e.g., the time it takes to pass through the user's gastrointestinal tract. Due to static nature of the programmed function of a typical ingestible computing device and encasement of the ingestible computing device by the body of the user, repurposing the ingestible computing device is oftentimes quite difficult or impossible.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of an ingestible computing device;

FIG. 2 is a simplified block diagram of at least one embodiment of an environment that may be established by the ingestible computing device of FIG. 1;

FIG. 3 is a simplified block diagram of at least one embodiment of a method for initializing device functions of the ingestible computing device of FIGS. 1 and 2;

FIG. 4 is a simplified block diagram of at least one embodiment of a method for adapting device functions that may be executed by the ingestible computing device of FIGS. 1 and 2; and

FIG. 5 is a simplified block diagram of at least one embodiment of a method for updating device functions that may be executed by the ingestible computing device of FIGS. 1 and 2.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C): (A and B); (B and C); (A and C); or (A, B, and C). Similarly, items listed in the form of “at least one of A, B, or C” can mean (A); (B); (C): (A and B); (B and C); (A or C); or (A, B, and C).

The disclosed embodiments may be implemented, in some cases, in hardware, firmware, software, or any combination thereof. The disclosed embodiments may also be implemented as instructions carried by or stored on one or more transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

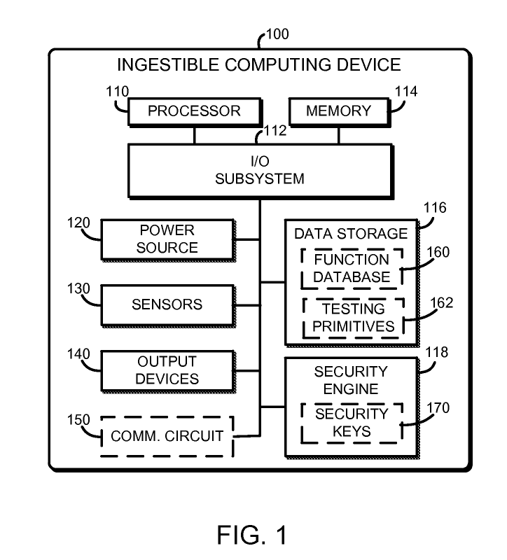

Referring now to FIG. 1, an illustrative ingestible computing device 100 is shown. The ingestible computing device 100 may be embodied as any type of computing device capable of being ingested into the body of a user. For example, the ingestible computing device may be configured to be swallowed, injected, surgically implanted, or otherwise placed into the body of the user. In use, the ingestible computing device 100 is configured to perform one or more device functions while located in the user's body. The particular function performed by the ingestible computing device 100 may be determined based on the original purpose of the ingestible computing device 100. For example, the initial device function of the ingestible computing device 100 may be to sense a biological stimuli of the user's body, respond to a biological stimuli of the user's body, sense an efficacy of a drug within the user's body, release a hormone into the user's body, produce a gene sequence within the user's body, record biological stimuli of the patient's body, generate an alert based on the sensed data, and/or perform any other function commonly performed by an ingestible computing device. Alternatively, in some embodiments, the ingestible computing device 100 may be configured to initially perform no device function.

Regardless of the initial device function, the ingestible computing device 100 is configured to monitor sensor data indicative of a biological characteristic of the user and adapt its initial (or subsequent) function based on the sensed biological characteristic. It should be appreciated that such function adaption occurs in-situ. That is, the ingestible computing device 100 is configured to adapt or reprogram its functions without removal from the user's body.

As discussed in more detail below, the ingestible computing device 100 is configured to adapt its device function by selection a function from a function database, obtain program code modules to implement the new function, and generate executable code using the program code modules. For example, the ingestible computing device 100 may compile the program code modules using a just-in-time compiler. The ingestible computing device 100 may subsequently execute the executable code to begin performing the new function.

The ingestible computing device 100 may be embodied as any type of ingestible computing device capable of performing the functions described herein. For example, the ingestible computing device 100 may be embodied as a smart pill, a smart implant, an artificial organ, or other computing device capable of ingestion or implantation into a user. As shown in FIG. 1, the illustrative ingestible computing device 100 includes a processor 110, an I/O subsystem 112, a memory 114, a data storage 116, a security engine 118, a power source 120, one or more sensors 130, one or more output devices 140, and a communication circuit 150 in some embodiments. Of course, the ingestible computing device 100 may include other or additional components, such as those commonly found in an ingestible device in other embodiments. Additionally, in some embodiments, one or more of the illustrative components may be incorporated in, or otherwise form a portion of, another component. For example, the memory 114, or portions thereof, may be incorporated in the processor 110 in some embodiments

The processor 110 may be embodied as any type of processor capable of performing the functions described herein. For example, the processor 110 may be embodied as a single or multi-core processor(s), a single or multi-socket processor, a digital signal processor, a microcontroller, or other processor or processing/controlling circuit. Similarly, the memory 114 may be embodied as any type of volatile or non-volatile memory or data storage capable of performing the functions described herein. In operation, the memory 114 may store various data and software used during operation of the ingestible computing device 100 such as operating systems, applications, programs, program code modules, libraries, and/or drivers. The memory 114 is communicatively coupled to the processor 110 via the I/O subsystem 112, which may be embodied as circuitry and/or components to facilitate input/output operations with the processor 110, the memory 114, and other components of the ingestible computing device 100. For example, the I/O subsystem 112 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 112 may form a portion of a system-on-a-chip (SoC) and be incorporated, along with the processor 110, the memory 114, and other components of the ingestible computing device 100, on a single integrated circuit chip.

The data storage 116 may be embodied as any type of device or devices configured for the short-term or long-term storage of data. For example, the data storage 116 may include any one or more memory devices and circuits, memory cards, hard disk drives, solid-state drives, or other data storage devices. Illustratively, the data storage 116 may include a function database 160 and/or testing primitives 162. The function database 160 includes data indicative of all the various device functions that ingestible computing device 102 can perform. For example, the function database 160 includes the program code modules required to perform each device function. Any one device function may correspond to one or multiple program code modules. As discussed in more detail below, the program code modules may be assembled and complied to generate executable code for causing the ingestible computing device 100 to perform the associated device function. The testing primitives of the data storage 116 may be embodied as any type of data usable to test the generated executable code prior to execution to ensure accuracy, expected behavior, and/or compatibility with the user.

The security engine 118 may be embodied as any type of processor or processing circuit capable of performing secure execution of instructions or providing security services for the ingestible computing device 100. For example, the security engine 108 may be embodied as a security co-processor, a trusted platform module (TPM), a manageability engine, a cryptographic accelerator processor, or the like. In some embodiments, the security engine 108 is embodied as an embedded out-of-band microprocessor, separate from processor 110, capable of executing code and addressing data inaccessible to the processor 110. Although shown in FIG. 1 as a separate component, the security engine 118 may form a portion of, or otherwise be embodied in, the processor 110, the I/O subsystem 112, or other component of the ingestible computing device 100. Illustratively, the security engine 118 stores a set of security keys, which may be embodied as cryptographic keys for authenticating the program code modules stored in the function database 160 and/or communications received from remote devices as discussed below.

The power source 120 may be embodied as any type of power source capable of providing power to the other components of the ingestible computing device 100. For example, the power source 120 may be embodied as a set of batteries or other type of power source.

The sensors 130 may be embodied as any type of sensors capable of sensing biological characteristics of the user. For example, the sensors 130 may be embodied as blood glucose sensors, cholesterol sensors, organ functionality sensors, or any other type of sensor capable of sensing a biological characteristic of the user. The sensors 130 may also be programmable. In some embodiments, the ingestible computing device 100 may include a large array of sensors 130, some of which may not be used when the ingestible computing device is programmed to perform certain functions.

The output devices 140 may be embodied as any type of output devices to facilitate the device functions of the ingestible computing device 100. For example, depending on the particular device function, the output devices 140 may facilitate the delivery of a drug to the user, the releasing of a hormone, wireless communication messages, the production of a gene sequence, and/or other functional output. Again, the ingestible computing device 100 may include a large array of output devices 140, some of which may not be used when the ingestible computing device is programmed to perform certain functions.

The communication circuit 122 may be embodied as any communication circuit, device, or collection thereof, capable of enabling communications between the ingestible computing device 100 and remote computing devices located outside of the user's body. To do so, the communication circuit 122 may be configured to use any one or more communication technology and associated protocols (e.g., Bluetooth®, Near Field Communication (NFC), body-coupled communication, Ethernet, Wi-Fi®, etc.) to effect such communication.

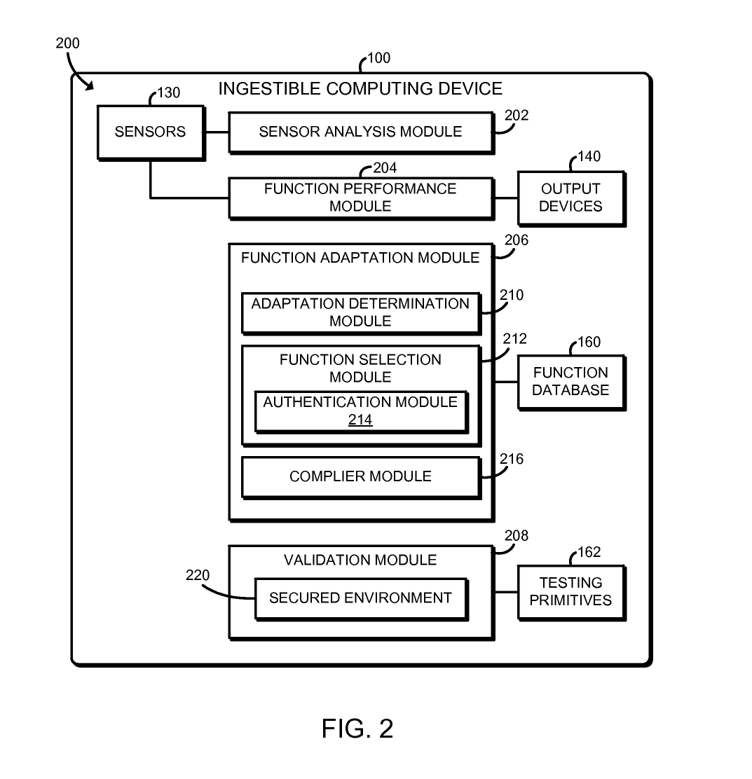

Referring now to FIG. 2, in use, the ingestible computing device 100 may establish an environment 200. The illustrative environment 200 includes a sensor analysis module 202, a function performance module 204, a function adaption module 206, and a validation module 208. Each of the modules and other components of the environment 200 may be embodied as firmware, software, hardware, or a combination thereof. For example the various modules, logic, and other components of the environment 200 may form a portion of, or otherwise be established by, the processor 110, the I/O subsystem 112, an SoC, or other hardware components of the ingestible computing device 100. As such, in some embodiments, any one or more of the modules of the environment 200 may be embodied as a circuit or collection of electrical devices (e.g., a sensor analysis circuit, a function performance circuit, a function adaption circuit, and a validation circuit, etc.).

The sensor analysis module 202 is configured to control the sensor 130 to generate sensor data indicative of a biological characteristic of the user. The particular biological characteristic or characteristics monitored by the sensor analysis module 202 may depend on the current device function of the ingestible computing device 100. For example, if the current device function is the production of insulin, the sensor analysis module 202 may monitor sensor data indicative of a blood glucose level of the user. Alternatively, if the device function is the delivery of a drug to the patient, the sensor analysis module 202 may be configured to monitor the sensor data indicative of the efficacy or levels of the delivered drug. The sensor analysis module 202 provides the generated sensor data to the function adaption module 206 for further analysis.

The function performance module 204 is configured to control components of the ingestible computing device 102 to perform the current device function. For example, the function performance module 204 may control one or more output devices 140 to perform the current device function (e.g., delivery of a drug, release of a hormone, production of a gene sequence, etc.). If the device function is related to sensing particular data, the function performance module 204 may also control one or more sensors 130 to perform the device function (e.g., sensing an efficacy of a drug within the user's body, recording biological stimuli or functionality, etc.).

The function adaption module 206 is configured to analyze the sensor data provided by the sensor analysis module 202, determine whether an adaption of the device function is required or desired based on the sensor data, and perform the adaption of the device function. For example, the sensor data may indicate the occurrence of an emergency condition (e.g., the user is having a heart attack), and the function adaption module 206 may determine that a new device function is required or desired to respond to the emergency condition (e.g., to release a drug to suppress the heart attack).

The function adaption module 206 includes an adaption determination module 210 configured to determine whether an adaption of the current device function is required based on the sensor data received from the sensor analysis module 202. To do so, the adaption determination module 210 may perform any suitable analysis or comparison on the sensor data. For example, in some embodiments, the sensor analysis module 202 may compare the sensor data to various threshold values to determine whether function adaption is required. It should be appreciated that the adaption of the device function may result in a completely different device function or a modification of the current device function. For example, the new device function may simply adjust the amount of delivery of a drug relative to the old device function, which performed the same function at a different delivery rate.

The function adaption module 206 also includes a function selection module 212 configured to select the new function from the function database 160 and retrieve the corresponding program code modules. The function selection module 212 may select the new function based on any criteria and using any suitable selection algorithm. In the illustrative embodiment, the function selection module 212 selects the new function based on the sensor data provided by the sensor analysis module 202. To select the new function, the function selection module 212 identifies the program code modules required to perform the new function and retrieve the program code modules from the function database 160. In some embodiments, the function selection module may include an authentication module 214 configured to authenticate the program code modules. For example, each program code module may be cryptographically signed, and the authentication module 214 may authenticate each program code using the security keys 170. Additionally, as discussed in more detail below, the ingestible computing device 100 may receive the new function (i.e., the program code modules to implement the new function) from a computing device external to the user's body. In such embodiments, the authentication module 214 may also authenticate the communication link, along with any received communication modules, using the security keys 170.

The ingestible computing device 100 generates executable code based on the program code modules, which may be executed by the ingestible computing device 100 to perform the new function. To do so, the ingestible computing device 100 includes a compiler module 216 to compile the program code modules. The compiler module 216 may be embodied as, or otherwise use, any type of complier. In one embodiment, the compiler module 216 is embodied as a just-in-time complier.

Before the new executable code is executed by the ingestible computing device 100, the executable code may be validated by the validation module 208 using the testing primitives 162. To do so, the validation module 208 may validate the executable code in a secured environment 220 (e.g., a trusted execution environment), which may be established by the security engine 118. The validation module 208 ensures the proper execution of the executable code and verifies the execution code performs the new function as desired. In some embodiments, the testing primitives 162 are configured based on the genetic makeup of the user, and the validation module 208 ensures the executable code performs the new function properly for that particular user. If the validation module 208 successfully validates the newly created executable code, the ingestible computing device 100 executes the new executable code and beings performing the new function. In doing so, the ingestible computing device 100 may or may not cease performing the previous function.

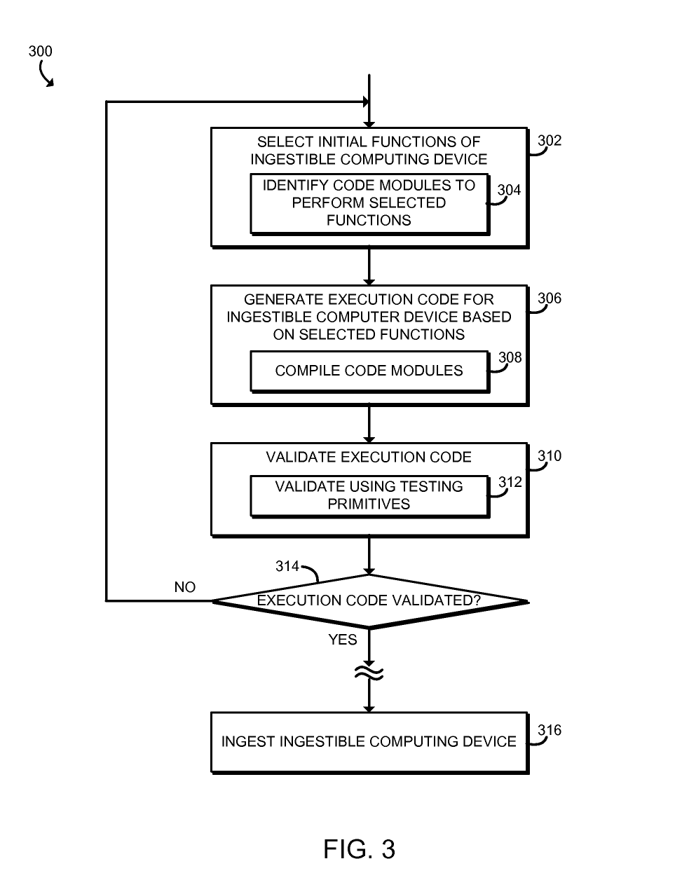

Referring now to FIG. 3, in use, the device function(s) of the ingestible computing device 100 may execute a method 300 for initializing device functions prior to ingestion of the device 100. The method 300 begins with block 302 in which the initial function(s) to be performed by the ingestible computing device 100 is selected. In some embodiments, the ingestible computing device 100 may select the initial functions (e.g., based on default choices) or a designer or manufacturer of the ingestible computing device 100 may select the initial functions. Regardless, in block 304, the program code modules required to perform the selection function or functions are identified.

Subsequently, in block 306, the executable code for performing the selected function is generated based on the identified program code modules. To do so, as discussed above, the program code modules are compiled in block 308 to generate the executable code. The generated executable code is subsequently validated in block 310. For example, the executable code may be validated in the secured environment 220 using the testing primitives 162 in block 312.

In block 314, the ingestible computing device 100 determines whether the execution code was successfully validated. If not, the method 300 loops back to block 302 in which a new initial function may be selected. If, however, the executable code was validated in block 314, the method 300 advances to block 316 in which the ingestible computing device 100 is ingested into the user's body. As discussed above, the ingestible computing device 100 may be ingested by the user by swallowing the ingestible computing device 100, injecting the ingestible computing device 100, implanting the ingestible computing device 100, or other methodology useable to place the ingestible computing device 100 within the user.

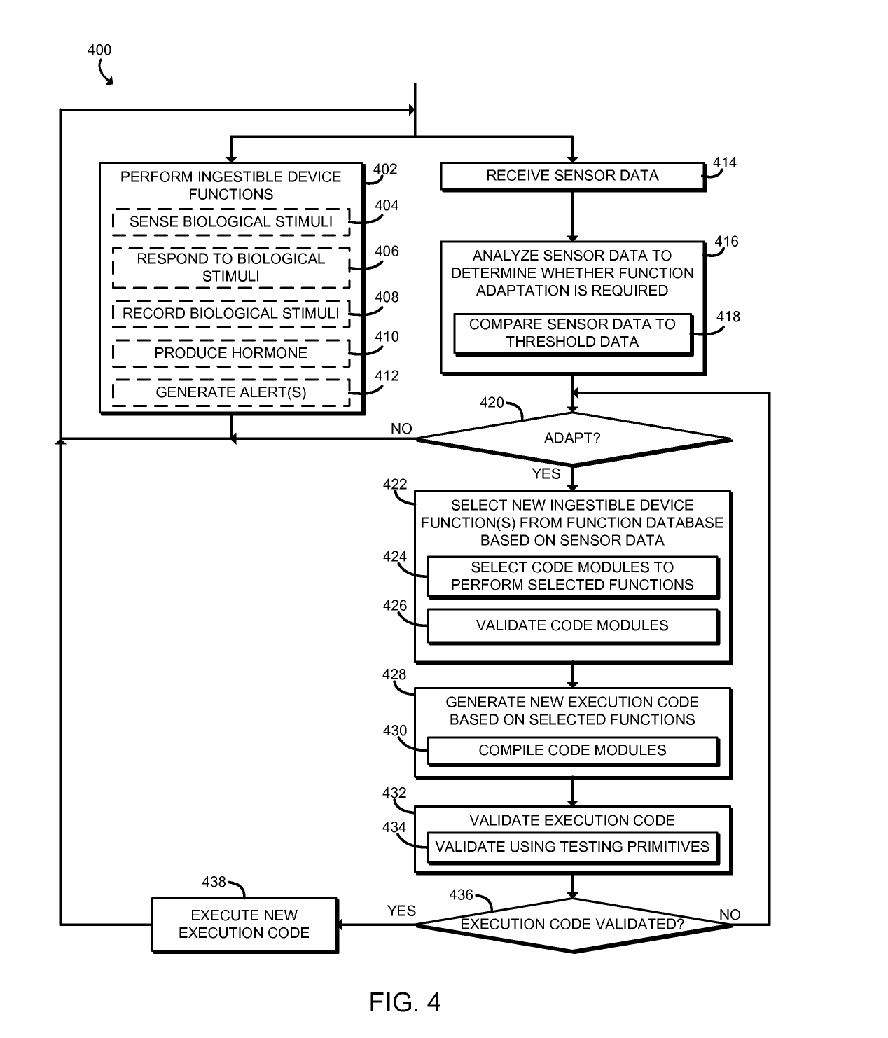

Referring now to FIG. 4, in use, the ingestible computing device 100 may perform a method 400 for adapting device functions of the ingestible computing device 100. The method 400 begins with blocks 402 and 414. In block 402, the ingestible computing device 100 performs the current device function. As discussed above, the ingestible computing device 100 may be configured to perform any type of device function typically performed by an ingestible computing device. For example, in block 404, the ingestible computing device 100 may sense biological stimuli of the user. For example, the ingestible computing device may sense the efficacy of a drug delivered to the user's body, the functionality of an organ of the user, the presence of a hormone or chemical in the user's body, and so forth. In block 406, the ingestible computing device 100 may respond to the sense biological stimuli of the user. For example, the ingestible computing device 100 may release a drug, produce a gene sequence, or the like. In block 408, the ingestible computing device 100 may record sensed biological stimuli (e.g., recording blood glucose levels). In block 410, the ingestible computing device 100 may produce a hormone (e.g., in response to sense biological stimuli). Additionally or alternatively, in block 412, the ingestible computing device 100 may generate one or more alerts. For example, the ingestible computing device 100 may generate the alert based on the sensed biological stimuli. The generated alerts may be embodied as local or remote alerts. For example, in some embodiments, the ingestible computing device 100 is configured to transmit an alert notification to a remote computing device in block 412.

Referring back to block 414, contemporaneously with the performance of its current device function or functions, the ingestible computing device 100 also receives sensor data from the sensors 130. The ingestible computing device 100 subsequently analyzes the sensor data to determine whether an adaption of the current device function of the ingestible computing device 100 is required or desired. To do so, for example, the ingestible computing device 100 may compare the received sensor data to threshold values or data (e.g., compare a blood glucose level to a threshold level).

In block 420, the ingestible computing device 100 determines whether to adapt the device function based on the analyzed sensor data in block 416. If not, the method 400 loops back to block 420 in which the ingestible computing device 100 continues to perform its current device function and block 414 in which ingestible computing device 100 continues monitoring the sensor data from the sensors 130. If, however, the ingestible computing device 100 determines that device function adaptation is required or desired, the method advances to block 422.

In block 422, the ingestible computing device 100 selects the new device function to be performed. As discussed above, the ingestible computing device 100 may select the new device function based on the sensed data from the sensors 130 or other criteria. In block 424, the ingestible computing device 100 identifies or selects the corresponding program codes from the function database 160 required to perform the selected device function. In block 426, the selected program codes may be validated using the security keys 170 as discussed above.

In block 428, the ingestible computing device 100 generates the new executable code based on the selected program code modules. To do so, the ingestible computing device 100 may compile the selected program code modules in block 430 (e.g., using a just-in-time complier). Subsequently, in block 432, the generated executable code is validated. For example, as discussed above, the generated executable code may be validated using the testing primitives 162. In block 436, the ingestible computing device 100 determines whether the newly generated executable code has been validated. If not, the method 400 loops back to block 420 in which the ingestible computing device 100 again determines whether device function adaption is required and may select a new function in block 422 if so. However, if the ingestible computing device 100 determines that the newly generated executable code is validated in block 436, the method 400 advances to block 438 in which the newly generated executable code is executed by the ingestible computing device 100, and the ingestible computing device 100 begins performing the device function in block 402.

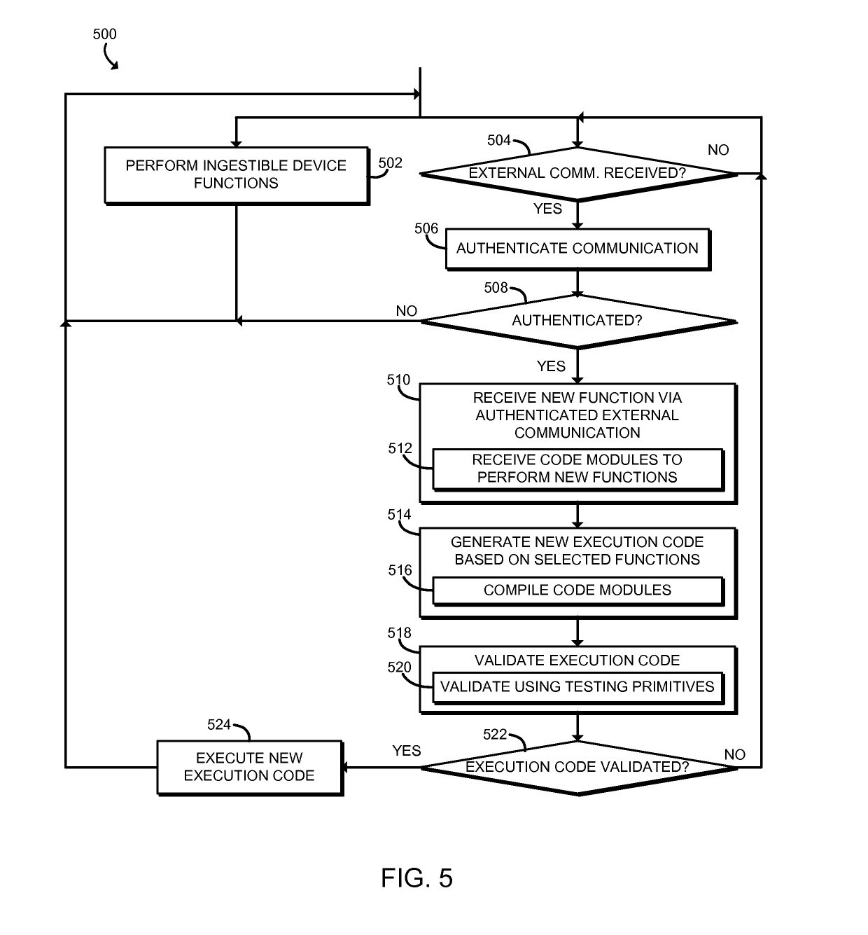

Referring now to FIG. 5, as discussed above, the ingestible computing device 100 may be configured to receive programming code modules for new functions from a remote computing device via wireless communication. In such embodiments, the ingestible computing device 100 may execute a method 500 for updating device functions of the ingestible computing device 100. The method 500 begins with block 502 and 504. In block 502, the ingestible computing device 100 performs the current device function as discussed above with regard to block 402 of method 400. In block 504, the ingestible computing device 100 determines whether an external communication has been received. If so, the method 500 advances to block 506 in which the ingestible computing device 100 authenticates the external communication. As discussed above, the ingestible computing device 100 may authenticate the external communication using the security keys 170. If the external communication is not authenticated, the method 500 loops back to block 502 in which the ingestible computing device 100 continues performing the current device function and block 504 in which the ingestible computing device 100 monitors for additional external communications.

If, however, the external communication is authenticated, the method 500 advances to block 510 in which the ingestible computing device 100 receives the new function via the external communication. To do so, the ingestible computing device 100 receives the program code modules used to perform the new function in block 512. Subsequently, in block 514, the ingestible computing device 100 generates a new executable code using the received program code modules. As discussed above, the ingestible computing device 100 may generate the new executable code by compiling the program code modules in block 516.

Subsequently, in block 518, the generated executable code is validated. For example, as discussed above, the generated executable code may be validated using the testing primitives 162 in block 520. In block 522, the ingestible computing device 100 determines whether the newly generated executable code has been validated. If not, the method 500 loops back to block 502 in which the ingestible computing device 100 continues performing the current device function and block 504 in which the ingestible computing device 100 monitors for additional external communications. However, if the ingestible computing device 100 determines that the newly generated executable code is validated in block 522, the method 500 advances to block 524 in which the newly generated executable code is executed by the ingestible computing device 100, and the ingestible computing device 100 begins performing the device function in block 502.

It should be appreciated that the technologies described herein provide a mechanism for updating the functionality for an ingestible computing device while remaining in the user's body. Such updating may occur in an autonomous manner as described above in regard to FIG. 4 or an assisted manner as described above in regard to FIG. 5.

### EXAMPLES

Illustrative examples of the devices, systems, and methods disclosed herein are provided below. An embodiment of the devices, systems, and methods may include any one or more, and any combination of, the examples described below.

Example 1 includes an ingestible computing device for performing a device function within the body of a user, the ingestible computing device comprising a sensor analysis module to generate sensor data indicative of a biological characteristic of a user of the ingestible computing device; a function performance module to perform the device function within a body of the user; and a function adaption module to determine whether adaption of the device function is required based on the sensor data and, in response to a determination that adaption of the device function is required, to (i) determine a new function to be performed by the ingestible computing device based on the sensor data; (ii) obtain one or more program code modules associated with the new function; (iii) generate executable code for the ingestible computing device based on the one or more program code modules, wherein the executable code causes the ingestible computing device to perform the new device function.

Example 2 includes the subject matter of Example 1, and wherein the sensor data comprises sensor data indicative of a biological function of the user's body.

Example 3 includes the subject matter of any of Examples 1 and 2, and wherein the sensor data comprises sensor data indicative of an efficacy of the device function.

Example 4 includes the subject matter of any of Examples 1-3, and wherein the sensor data comprises sensor data indicative of an efficacy of a drug within the user's body.

Example 5 includes the subject matter of any of Examples 1-4, and wherein to perform the device function comprises to perform at least one of (i) sensing a biological stimuli of the user's body, (ii) responding to a biological stimuli of the user's body, (iii) sensing an efficacy of a drug within the user's body, (iv) releasing a hormone into the user's body, (v) producing a gene sequence within the user's body, (vi) recording biological stimuli of the patient's body, or (vii) generating an alert based on the sensor data.

Example 6 includes the subject matter of any of Examples 1-5, and wherein to determine whether adaption of the device function is required comprises to compare the sensor data to a threshold and determine that adaption of the device function is required in response the sensor data having a reference relationship to the threshold.

Example 7 includes the subject matter of any of Examples 1-6, and further including a function database having stored therein available functions of the ingestible computing device, wherein to determine the new function to be performed by the ingestible computing device comprises to select the new function from the function database.

Example 8 includes the subject matter of any of Examples 1-7, and wherein to obtain the one or more program code modules comprises to retrieve the one or more program code modules from the function database based on the selected new function.

Example 9 includes the subject matter of any of Examples 1-8, and further including a validation module to validate the one or more program codes based on a set of testing primitives stored on the ingestible computing device and prior to the generation of the executable code.

Example 10 includes the subject matter of any of Examples 1-9, and wherein each of the one or more program code modules is customized for the particular user based on a biological sample of the user.

Example 11 includes the subject matter of any of Examples 1-10, and wherein to generate the executable code for the ingestible computing device comprises to compile the one or more program code modules to generate the executable code.

Example 12 includes the subject matter of any of Examples 1-11, and further including a validation module to validate the executable code based on a set of testing primitives stored on the ingestible computing device.

Example 13 includes the subject matter of any of Examples 1-12, and further including a security engine having stored therein a set of cryptographic keys, wherein the security engine is to validate the executable code based on the set of cryptographic keys.

Example 14 includes the subject matter of any of Examples 1-13, and wherein the function performance module is further to execute, in response to validation of the executable code, the executable code to cause the ingestible computing device to perform the new device function.

Example 15 includes the subject matter of any of Examples 1-14, and further including a communication module to receive the one or more program code modules via a transmission from a computing device located externally to the user's body.

Example 16 includes the subject matter of any of Examples 1-15, and further including a security engine having stored therein a set of cryptographic keys, wherein the security engine is to authenticate, prior to the generation of the executable code, the transmission using the cryptographic keys.

Example 17 includes the subject matter of any of Examples 1-16, and, wherein to generate the executable code for the ingestible computing device comprises to compile the one or more program code modules to generate the executable code.

Example 18 includes the subject matter of any of Examples 1-17, and further including a validation module to validate the executable code based on a set of testing primitives stored on the ingestible computing device.

Example 19 includes a method for managing a device function of an ingestible computing device, the method comprising generating, by a sensor of the ingestible computing device, senor data indicative of a biological characteristic of a user of the ingestible computing device; performing, by the ingestible computing device, the device function within a body of the user; determining, by the ingestible computing device, whether adaption of the device function is required based on the sensor data; and in response to a determination that adaption of the device function is required (i) determining, by the ingestible computing device, a new function to be performed by the ingestible computing device based on the sensor data; (ii) obtaining, by the ingestible computing device, one or more program code modules associated with the new function; (iii) generating, by the ingestible computing device, executable code for the ingestible computing device based on the one or more program code modules, wherein the executable code causes the ingestible computing device to perform the new device function.

Example 20 includes the subject matter of Example 19, and wherein generating the sensor data comprises generating sensor data indicative of a biological function of the user's body.

Example 21 includes the subject matter of any of Examples 19 and 20, and wherein generating the sensor data comprises generating sensor data indicative of an efficacy of the device function.

Example 22 includes the subject matter of any of Examples 19-21, and wherein generating the sensor data comprises generating sensor data indicative of an efficacy of a drug within the user's body.

Example 23 includes the subject matter of any of Examples 19-22, and wherein performing the device function comprises performing at least one of (i) sensing a biological stimuli of the user's body, (ii) responding to a biological stimuli of the user's body, (iii) sensing an efficacy of a drug within the user's body, (iv) releasing a hormone into the user's body, (v) producing a gene sequence within the user's body, (vi) recording biological stimuli of the patient's body, and (vii) generating an alert based on the sensor data.

Example 24 includes the subject matter of any of Examples 19-23, and wherein determining whether adaption of the device function is required comprises comparing the sensor data to a threshold and determining that adaption of the device function is required in response the sensor data having a reference relationship to the threshold.

Example 25 includes the subject matter of any of Examples 19-24, and wherein determining the new function to be performed by the ingestible computing device comprises selecting the new function from a function database of available functions stored on the ingestible computing device.

Example 26 includes the subject matter of any of Examples 19-25, and wherein obtaining the one or more program code modules comprises retrieving the one or more program code modules from the function database based on the selected new function.

Example 27 includes the subject matter of any of Examples 19-26, and further including validating, by the ingestible computing device, the one or more program codes based on a set of testing primitives stored on the ingestible computing device and prior to the generation of the executable code.

Example 28 includes the subject matter of any of Examples 19-27, and wherein each of the one or more program code modules are customized for the particular user based on a biological sample of the user.

Example 29 includes the subject matter of any of Examples 19-28, and wherein generating the executable code for the ingestible computing device comprises compiling the one or more program code modules to generate the executable code.

Example 30 includes the subject matter of any of Examples 19-29, and further including validating, by the ingestible computing device, the executable code based on a set of testing primitives stored on the ingestible computing device.

Example 31 includes the subject matter of any of Examples 19-30, and wherein validating the executable code comprises validating the executable code based on a set of cryptographic keys stored in a security engine of the ingestible computing device.

Example 32 includes the subject matter of any of Examples 19-31, and further including executing, by the ingestible computing device and in response to validation of the executable code, the executable code to cause the ingestible computing device to perform the new device function.

Example 33 includes the subject matter of any of Examples 19-32, and wherein obtaining one or more program code modules comprises receiving, by the ingestible computing device, the one or more program code via a transmission from a computing device located externally to the user's body.

Example 34 includes the subject matter of any of Examples 19-33, and further including authenticating, by the ingestible computing device and prior to generating of the executable code, the transmission using cryptographic keys stored in a security engine of the ingestible computing device.

Example 35 includes the subject matter of any of Examples 19-34, and wherein generating the executable code for the ingestible computing device comprises compiling the one or more program code modules to generate the executable code.

Example 36 includes the subject matter of any of Examples 19-35, and further including validating, by the ingestible computing device, the executable code based on a set of testing primitives stored on the ingestible computing device.

Example 37 includes one or more computer-readable storage media comprising a plurality of instructions stored thereon that, in response to execution, cause a computing device to perform the method of any of claims 19-36.

Example 38 includes a computing system for managing security threats, the computing device comprising means for performing the method of any of Examples 19-36.

1-25. (canceled)

26. An ingestible computing device for performing a device function within the body of a user, the ingestible computing device comprising:
a sensor analysis module to generate sensor data indicative of a biological characteristic of a user of the ingestible computing device;
a function performance module to perform the device function within a body of the user; and
a function adaption module to determine whether adaption of the device function is required based on the sensor data and, in response to a determination that adaption of the device function is required, to:
(i) determine a new function to be performed by the ingestible computing device based on the sensor data;
(ii) obtain one or more program code modules associated with the new function;
(iii) generate executable code for the ingestible computing device based on the one or more program code modules, wherein the executable code causes the ingestible computing device to perform the new device function.

27. The ingestible computing device of claim 26, further comprising a function database having stored therein available functions of the ingestible computing device,
wherein to determine the new function to be performed by the ingestible computing device comprises to select the new function from the function database.

28. The ingestible computing device of claim 27, wherein to obtain the one or more program code modules comprises to retrieve the one or more program code modules from the function database based on the selected new function.

29. The ingestible computing device of claim 28, further comprising a validation module to validate the one or more program codes based on a set of testing primitives stored on the ingestible computing device and prior to the generation of the executable code.

30. The ingestible computing device of claim 29, wherein each of the one or more program code modules is customized for the particular user based on a biological sample of the user.

31. The ingestible computing device of claim 26, wherein to generate the executable code for the ingestible computing device comprises to compile the one or more program code modules to generate the executable code.

32. The ingestible computing device of claim 31, further comprising a validation module to validate the executable code based on a set of testing primitives stored on the ingestible computing device.

33. The ingestible computing device of claim 32, wherein the function performance module is further to execute, in response to validation of the executable code, the executable code to cause the ingestible computing device to perform the new device function.

34. The ingestible computing device of claim 26, further comprising a communication module to receive the one or more program code modules via a transmission from a computing device located externally to the user's body.

35. One or more computer-readable storage media comprising a plurality of instructions stored thereon that, in response to execution, cause a ingestible computing device to:
generate, by a sensor of the ingestible computing device, senor data indicative of a biological characteristic of a user of the ingestible computing device;
perform the device function within a body of the user;
determine whether adaption of the device function is required based on the sensor data; and
in response to a determination that adaption of the device function is required:
(i) determine a new function to be performed by the ingestible computing device based on the sensor data;
(ii) obtain one or more program code modules associated with the new function;
(iii) generate executable code for the ingestible computing device based on the one or more program code modules, wherein the executable code causes the ingestible computing device to perform the new device function.

36. The one or more computer-readable storage media of claim 35, wherein to determine the new function to be performed by the ingestible computing device comprises to select the new function from a function database of available functions stored on the ingestible computing device.

37. The one or more computer-readable storage media of claim 36, wherein to obtain the one or more program code modules comprises to retrieve the one or more program code modules from the function database based on the selected new function.

38. The one or more computer-readable storage media of claim 37, wherein the plurality of instructions further cause the ingestible computing device to validate the one or more program codes based on a set of testing primitives stored on the ingestible computing device and prior to the generation of the executable code.

39. The one or more computer-readable storage media of claim 37, wherein each of the one or more program code modules are customized for the particular user based on a biological sample of the user.

40. The one or more computer-readable storage media of claim 35, wherein to generate the executable code for the ingestible computing device comprises to compile the one or more program code modules to generate the executable code.

41. The one or more computer-readable storage media of claim 40, wherein the plurality of instructions further cause the ingestible computing device to:
validate the executable code based on a set of testing primitives stored on the ingestible computing device; and
execute, in response to validation of the executable code, the executable code to cause the ingestible computing device to perform the new device function.

42. The one or more computer-readable storage media of claim 40, wherein to obtain one or more program code modules comprises to receive the one or more program code modules via a transmission from a computing device located externally to the user's body.

43. A method for managing a device function of an ingestible computing device, the method comprising:
generating, by a sensor of the ingestible computing device, senor data indicative of a biological characteristic of a user of the ingestible computing device;
performing, by the ingestible computing device, the device function within a body of the user;
determining, by the ingestible computing device, whether adaption of the device function is required based on the sensor data; and
in response to a determination that adaption of the device function is required:
(i) determining, by the ingestible computing device, a new function to be performed by the ingestible computing device based on the sensor data;
(ii) obtaining, by the ingestible computing device, one or more program code modules associated with the new function;
(iii) generating, by the ingestible computing device, executable code for the ingestible computing device based on the one or more program code modules, wherein the executable code causes the ingestible computing device to perform the new device function.

44. The method of claim 43, wherein determining the new function to be performed by the ingestible computing device comprises selecting the new function from a function database of available functions stored on the ingestible computing device.

45. The method of claim 44, wherein obtaining the one or more program code modules comprises retrieving the one or more program code modules from the function database based on the selected new function.

46. The method of claim 45, further comprising validating, by the ingestible computing device, the one or more program codes based on a set of testing primitives stored on the ingestible computing device and prior to the generation of the executable code.

47. The method of claim 45, wherein each of the one or more program code modules are customized for the particular user based on a biological sample of the user.

48. The method of claim 43, wherein generating the executable code for the ingestible computing device comprises compiling the one or more program code modules to generate the executable code.

49. The method of claim 48, further comprising:
validating, by the ingestible computing device, the executable code based on a set of testing primitives stored on the ingestible computing device; and
executing, by the ingestible computing device and in response to validation of the executable code, the executable code to cause the ingestible computing device to perform the new device function.

50. The method of claim 43, wherein obtaining one or more program code modules comprises receiving, by the ingestible computing device, the one or more program code modules via a transmission from a computing device located externally to the user's body.
